(19) United States
(12) Patent Application Publication
(10) Pub. No.: US 2021/0121470 A1
(43) Pub. Date: Apr. 29, 2021

(54) METHOD OF TREATING MALIGNANT RHABDOID TUMOR OF THE OVARY (MRTO)/SMALL CELL CANCER OF THE OVARY OF THE HYPERCALCEMIC TYPE(SCCOHT) WITH AN EZH2 INHIBITOR

(71) Applicant: Epizyme, Inc., Cambridge, MA (US)

(72) Inventor: Heike KEILHACK, Belmont, MA (US)

(21) Appl. No.: 17/011,132

(22) Filed: Sep. 3, 2020

Related U.S. Application Data
(63) Continuation of application No. 16/593,010, filed on Oct. 4, 2019, now abandoned.
Application No. 16/593,010 is a continuation of application No. 15/762,839, filed on Mar. 23, 2018, now abandoned.
(60) Provisional application No. 62/252,188, filed on Nov. 6, 2015.
(60) Provisional application No. 62/233,146, filed on Sep. 25, 2015.

(51) Int. Cl. A61K 31/5377; A61K 31/445; A61K 31/496; A61K 31/4412; A61K 31/501; A61P 35/00; A61K 31/44; A61K 31/4545

(52) U.S. Cl. CPC A61K 31/5377; A61K 31/445; A61K 31/496; A61K 31/4545; A61K 31/501; A61P 35/00; A61K 31/44; A61K 31/4412

(57) ABSTRACT

The disclosure provides a method of treating a malignant rhabdoid tumor in a subject in need thereof including administering to the subject a therapeutically-effective amount of an enhancer of a zeste homolog 2 (EZH2) inhibitor. In certain embodiments of this method the malignant rhabdoid tumor is small cell cancer of the ovary of the hypercalcemic type (SCCOHT) and the EZH2 inhibitor is tazemetostat (also known as Tazemetostat).

### RELATED APPLICATIONS

This application is a continuation of U.S. application Ser. No. 16/593,010, filed on Oct. 4, 2019, which is a continuation of U.S. application Ser. No. 15/762,839, filed on Mar. 23, 2018, which is a U.S. National Phase application, filed under 35 U.S.C. § 371, of International Application No. PCT/US2016/053673, filed on Sep. 26, 2016, which claims priority to, and the benefit of, U.S. Provisional Application Nos. 62/233,146, filed Sep. 25, 2015, and 62/252,188, filed Nov. 6, 2015, the contents of each of which are incorporated herein by reference in their entireties.

### FIELD OF THE DISCLOSURE

The disclosure is directed to the fields of small molecule therapies, cancer, and methods of treating rare cancer types.

### BACKGROUND

There is a long-felt yet unmet need for effective treatments for certain cancers caused by genetic alterations or loss of function of subunits of the SWI/SNF chromatin remodeling complex that result in EZH2-dependent oncogenesis.

### SUMMARY

The disclosure provides effective treatments for INI1-negative and SMARCA4-negative tumors, such as malignant rhabdoid tumors (MRTs) and epithelioid sarcoma. INI1 and SMARCA4 are critical proteins of the SWItch/Sucrose NonFermentable (SWI/SNF) chromatin remodeling complex, which opposes the activity of EZH2. Genetic alterations or loss of function of either can result in EZH2-dependent oncogenesis in certain cancer backgrounds, thus rendering these tumors sensitive to EZH2 inhibition. In certain embodiments MRTs can be INI1-negative, INI1-deficient, SMARCA4-negative, SMARCA4 deficient, SMARCA2 negative, SMARCA2 deficient, or comprise a mutation on one or more other components of the SWI/SNF complex.

In certain embodiments of the disclosure the MRT is malignant rhabdoid tumor of the ovary (MRTO), also referred to as small cell cancer of the ovary of the hypercalcemic type (SCCOHT). The disclosure provides a method of treating SCCOHT in a subject in need thereof comprising administering to the subject a therapeutically-effective amount of an EZH2 inhibitor, e.g., tazemetostat (EPZ-6438). In some embodiments, the EZH2 inhibitor, e.g., tazemetostat, is formulated as an oral tablet. In some embodiments, the therapeutically effective amount of the EZH2 inhibitor, e.g., tazemetostat, is about 800 mg/kg. In some embodiments, the EZH2 inhibitor, e.g., tazemetostat, is administered twice per day.

In certain embodiments of the disclosure the MRT is epithelioid sarcoma. The disclosure provides a method of treating epithelioid sarcoma in a subject in need thereof comprising administering to the subject a therapeutically-effective amount of an EZH2 inhibitor, e.g., tazemetostat (EPZ-6438). In some embodiments, the EZH2 inhibitor, e.g., the tazemetostat, is formulated as an oral tablet. In some embodiments, the therapeutically effective amount of the EZH2 inhibitor, e.g., tazemetostat, is about 800 mg/kg. In some embodiments, the EZH2 inhibitor, e.g., tazemetostat, is administered twice per day.

According to the methods of the disclosure, the EZH2 inhibitor inhibits tri-methylation of lysine 27 of histone 3 (H3K27). In certain embodiments, the EZH2 inhibitor of the disclosure may comprise, consist essentially of or consist of:

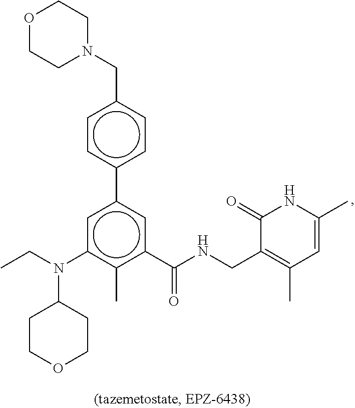

or a pharmaceutically acceptable salt thereof.

EZH2 inhibitors of the disclosure may be administered orally. In certain embodiments, EZH2 inhibitors may be formulated as an oral tablet.

Methods of the disclosure for treating cancer in a subject in need thereof comprise administering a therapeutically-effective amount of an EZH2 inhibitor to the subject. In certain embodiments, the therapeutically-effective amount of the EZH2 inhibitor is a dose of between 10 mg/kg/day and 1600 mg/kg/day, inclusive of the endpoints. Therefore, in certain embodiments of these methods, the EZH2 inhibitor is administered at a dose of between 10 mg/kg/day and 1600 mg/kg/day, inclusive of the endpoints. In certain embodiments, the therapeutically-effective amount of the EZH2 inhibitor is a dose of about 100, 200, 400, 800, or 1600 mg. Therefore, in certain embodiments of these methods, the EZH2 inhibitor is administered at a dose of about 100, 200, 400, 800, or 1600 mg. In certain embodiments, the therapeutically-effective amount of the EZH2 inhibitor is a dose of about 800 mg. Therefore, in certain embodiments of these methods, the EZH2 inhibitor is administered at a dose of about 800 mg. In certain embodiments, a therapeutically-effective amount of an EZH2 inhibitor may be administered to the subject twice per day (BID).

Methods of the disclosure for treating cancer including treating a malignant rhabdoid tumor (MRT). In preferred embodiments, methods of the disclosure are used to treat a subject having a malignant rhabdoid tumor of the ovary (MRTO). MRTO may also be referred to as small cell cancer of the ovary of the hypercalcemic type (SCCOHT). In certain embodiments, the MRTO or SCCOHT and/or the subject are characterized as SMARCA4-negative, SMARCA4 deficient, SMARCA2 negative, SMARCA2 deficient, or as having a mutation or deficiency in one or more other components of the SWI/SNF complex. In certain embodiments, the MRTO or SCCOHT and/or the subject are characterized as SMARCA4-negative. In certain embodiments, the MRTO or SCCOHT and/or the subject are characterized as SMARCA4-negative or SMARCA4-deficient; and SMARCA2-negative or SMARCA2-deficient. As used herein SMARCA4-negative and/or SMARCA4-deficient cells may contain a mutation in the SMARCA4 gene, corresponding SMARCA4 transcript (or cDNA copy thereof), or SMARCA4 protein, that prevents transcription of a SMARCA4 gene, translation of a SMARCA4 transcript, and/or decreases/inhibits an activity of a SMARCA4 protein. As used herein SMARCA4-negative cells may contain a mutation in the SMARCA4 gene, corresponding SMARCA4 transcript (or cDNA copy thereof), or SMARCA4 protein that prevents transcription of a SMARCA4 gene, translation of a SMARCA4 transcript, and/or decreases/inhibits an activity of a SMARCA4 protein.

Methods of the disclosure for treating cancer including treating a malignant rhabdoid tumor (MRT). In some preferred embodiments, methods of the disclosure are used to treat a subject having an epithelioid sarcoma. In certain embodiments, the epithelioid sarcoma is characterized as SMARCA4-negative, SMARCA4 deficient, SMARCA2 negative, SMARCA2 deficient, or as having a mutation or deficiency in one or more other components of the SWI/SNF complex. In certain embodiments, the epithelioid sarcoma and/or the subject are characterized as SMARCA4-negative. In certain embodiments, the epithelioid sarcoma and/or the subject are characterized as SMARCA4-negative or SMARCA4-deficient; and SMARCA2-negative or SMARCA2-deficient.

Methods of the disclosure may be used to treat a subject who is SMARCA4-negative or who has one or more cells that may be SMARCA4-negative. SMARCA4 expression and/or SMARCA4 function may be evaluated by fluorescent and non-fluorescent immunohistochemistry (IHC) methods, including well known to one of ordinary skill in the art. In a certain embodiment the method comprises: (a) obtaining a biological sample from the subject; (b) contacting the biological sample or a portion thereof with an antibody that specifically binds SMARCA4; and (c) detecting an amount of the antibody that is bound to SMARCA4. Alternatively, or in addition, SMARCA4 expression and/or SMARCA4 function may be evaluated by a method comprising: (a) obtaining a biological sample from the subject; (b) sequencing at least one DNA sequence encoding a SMARCA4 protein from the biological sample or a portion thereof; and (c) determining if the at least one DNA sequence encoding a SMARCA4 protein contains a mutation affecting the expression and/or function of the SMARCA4 protein. SMARCA4 expression or a function of SMARCA4 may be evaluated by detecting an amount of the antibody that is bound to SMARCA4 and by sequencing at least one DNA sequence encoding a SMARCA4 protein, optionally, using the same biological sample from the subject.

Subjects of the disclosure may be female. Subjects of the disclosure may be less than 40, 30, or 20 years of age. In certain embodiments, subjects of the disclosure may be between 20 and 30 years of age, inclusive of the endpoints.

As used herein, the term “treating” may comprise preventing and/or inhibiting proliferation of a cancer cell, including, but not limited to a MRTO/SCCOHT cell.

### BRIEF DESCRIPTION OF THE DRAWINGS

FIG. 1 is a schematic depiction of EZH2-mediated methylation of H3K27me3, an epigenetic modification that represses gene transcription.

FIG. 2 is a schematic depiction of an antagonism of PRC2 and SWI-SNF-dependent chromatin remodeling that regulates pluripotency.

FIG. 3 is a schematic depiction of the normal downregulation of EZH2 as progenitor cells become differentiated.

FIG. 4A is a schematic depiction of INI1 (SMARCB2)-mediated oncogenic dependency on EZH2 in tumor cells.

FIG. 4B is a graph showing that EZH2 knockout reverses oncogenesis induced by INI1 loss. Exemplary INI1-deficient tumors include, but are not limited to, malignant rhabdoid tumor and epithelial sarcoma.

FIG. 5A is a photograph of an immunohistochemistry procedure depicting expression of INI1 in MRTO/SCCOHT.

FIG. 5B is a photograph of an immunohistochemistry procedure depicting a loss of expression of SMARCA4 in MRTO/SCCOHT.

FIG. 6A is a series of x-ray films of a 27 year old female with SMARCA4-negative MRTO/SCCOHT at baseline (left), after 8 weeks of treatment with EPIZ-6438 (Tazemetostat) twice daily at a dosage of 1600 mg.

FIG. 6B is a schematic depiction of the course of treatment for the subject treated in FIG. 6A.

FIG. 7A is an x-ray film of a malignant rhabdoid tumor (MRT) in an infant. MRTs are pediatric, however adult cases have been reported. MRTs often occur in the kidney, CNS and soft tissue. Importantly, MRTs are often chemo-resistant leading to a dismal prognosis with survival rates of less than 25%.

FIG. 7B is a graph depicting the proportion of subjects alive as a function of time (months) after diagnosis of an INI1-negative rhabdoid tumor.

FIG. 7C is a graph depicting the percentage of subjects alive as a function of time (months) after diagnosis of an INI1-negative rhabdoid tumor.

FIG. 8A is a chemical structure diagram of tazemetostat.

FIG. 8B is a pair of schematic diagrams depicting the relative selectivity of tazemetostat for EZH2.

FIG. 8C is a graph demonstrating the antitumor activity of tazemetostat treatment in a xenograft model of INI1-negative MRT (G401).

FIG. 9 is a series of photographs of IHC depicting EZH2 target inhibition in tumor tissue before and after administration of tazemetostat.

FIG. 10 is a graph depicting the best response in patients with solid tumors.

FIG. 11 is a series of photographs depicting the complete remission (CR) of an INI1-negative malignant rhabdoid tumor in a 55 year old male undergoing treatment with tazemetostat at a dose of 800 mg BID.

FIG. 12 is a series of photographs depicting the partial remission (PR) of an INI1-negative epithelioid sarcoma in a 44 year old male undergoing treatment with tazemetostat at a dose of 800 mg BID.

FIG. 13A is a chemical structure diagram of Compound D.

FIG. 13B is a pair of graphs depicting the results of a long term 2D proliferation assay for Compound D in SMARCA4 and ARID1A ovarian cell lines. The day 14 IC₅₀ values are shown. SMARCA4-negative cell lines show anti-proliferative effects with EZH2 inhibitor Compound D, ARID1A mutated ovarian cell lines do not.

FIG. 13C is a graph displaying the results of a 14 day proliferation study with Compound D in the SMARCA4- and SMARCA2-negative small cell carcinoma of the ovary, hypercalcemic type (SCCOHT) cell line Bin-67. Growth curves are shown for 8 different treatment conditions ranging from 0.01-10 μM. The day 14 IC₅₀ value is 10 nM.

FIG. 13D is a Western Blot demonstrating the reduction of H3K27me3 levels in Compound D-treated Bin-67 cells on day 14. H3K27me3 levels were completely reduced on day 14 at all concentrations of Compound D.

FIG. 13E is a series of graphs illustrating the 3D growth effects of ARID1A-mutated ovarian cell lines treated with Compound D. No effects were observed with Compound D after 14 days. 3D assays were performed using the Scivax Nanoculture technology whereby the micro-patterned scaffold mimics the ECM.

FIG. 14 is a Western Blot analysis of the characterization of SMARCA2 and SMARCA4 loss in ovarian cell line panels. Protein levels of SMARCA2, SMARCB1, and SMARCA4 were evaluated in 30 ovarian cell lines. Two misdiagnosed SCCOHT cell lines (TOV112D, COV434) were identified based on the dual loss of SMARCA2 and SMARCA4 expression. Mutations were taken from CCLE and COSMIC databases.

FIG. 15 is an immunohistochemical analysis of core SWI/SNF proteins in SCCOHT, showing dual loss of SMARCA4/BRG1 and SMARCA2/BRM in SCCOHT. Endothelium and lymphocytes are internal positive controls for both proteins. Arrows denote rare tumor cells expressing SMARCA2. SMARCB1/INI1 protein expression serves as a positive control for tumor cell immunoreactivity (see, e.g., Karnezis et al. J. Pathol 2016; 238: 389-400.

FIG. 16 is a graph showing CRISPR pooled screen data from almost 100 cell lines, including four ovarian cell lines. The ordinate represents the RSA (Redundant siRNA activity) score which characterizes the sensitivity of knockout to EZH2. COV434 was identified to be of SCCOHT origin based on dual loss of SMARCA2 and SMARCA4, and was the only ovarian cell line to be sensitive to EZH2 knockout.

FIG. 17A is a graph illustrating results from long-term proliferation assays of ovarian cell lines treated with tazemetostat.

FIG. 17B is a graph showing dose-dependent inhibition of cell growth in SMARCA2-deficient and SMARCA4-deficient cell lines upon treatment with tazemetostat.

FIG. 18A is a graph illustrating tumor growth inhibition and terminal tumor volume in an in vivo SCCOHT xenograft model (Bin-76) after 18 days of treatment with tazemetostat.

FIG. 18B is a graph illustrating reduction of H3K27me3 in Bin-67 xenograft tumors after 18 days of treatment with tazemetostat.

FIG. 19A is a graph illustrating tumor growth inhibition and terminal tumor volume in an in vivo SCCOHT xenograft model (COV434) after 28 days of treatment with tazemetostat.

FIG. 19B is a graph illustrating reduction of H3K27me3 in COV434 xenograft tumors after 28 days of treatment with tazemetostat.

FIG. 20A is a graph illustrating tumor growth inhibition and terminal tumor volume in an in vivo SCCOHT xenograft model (TOV112D) after 14 days of treatment with tazemetostat.

FIG. 20B is a graph illustrating reduction of H3K27me3 in TOV112D xenograft tumors after 14 days of treatment with tazemetostat.

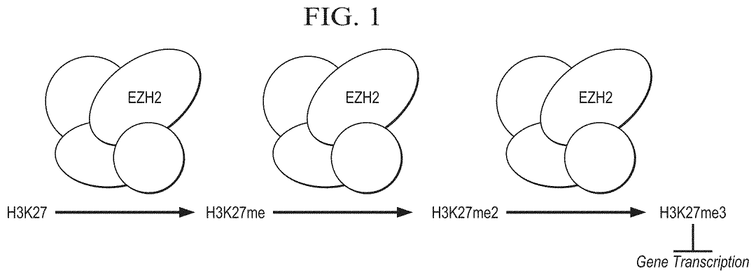

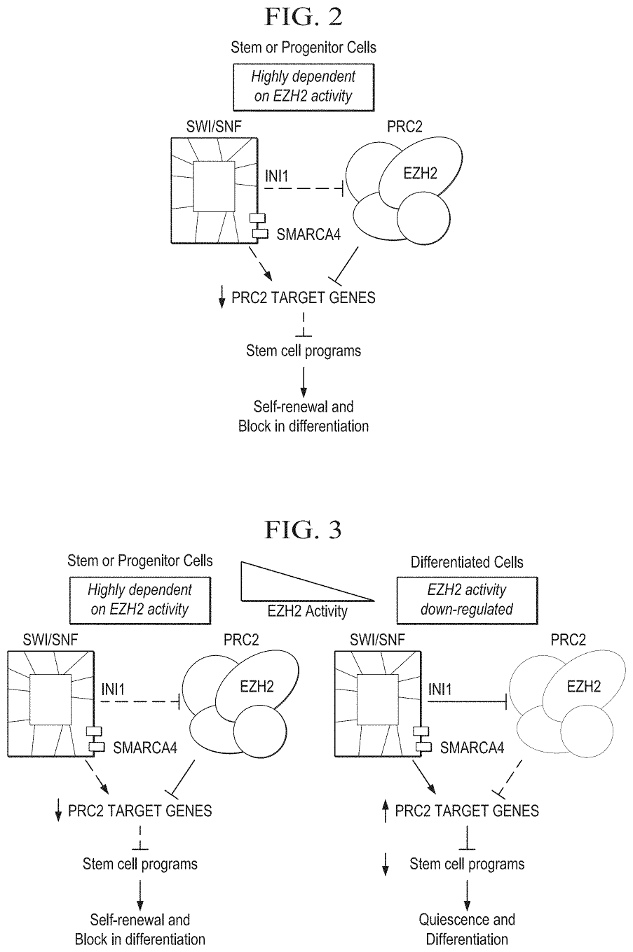

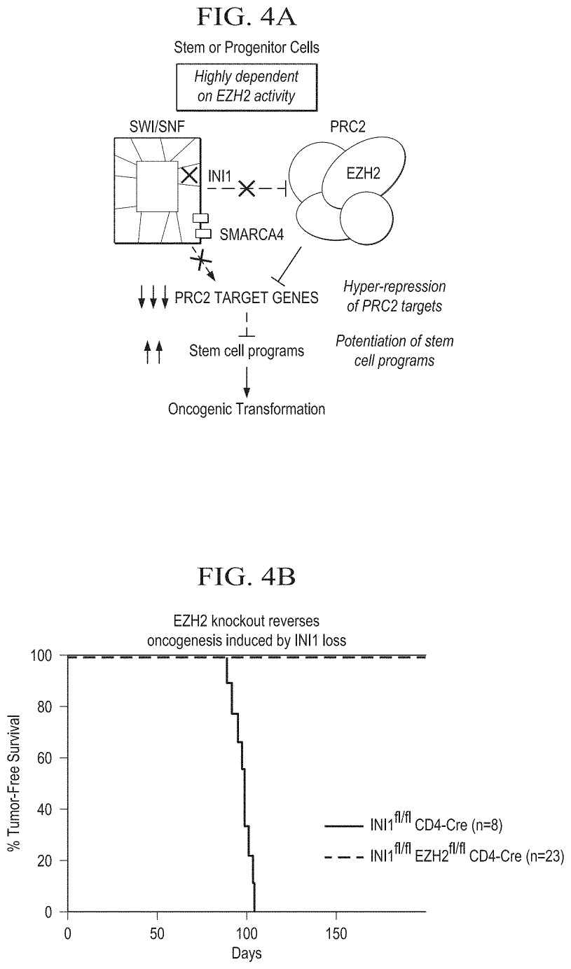

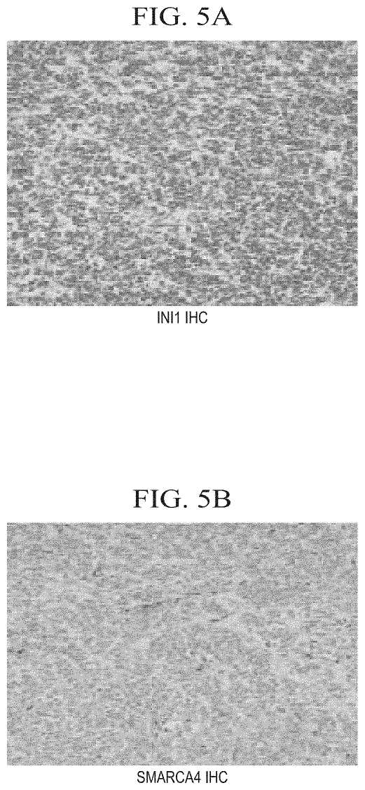

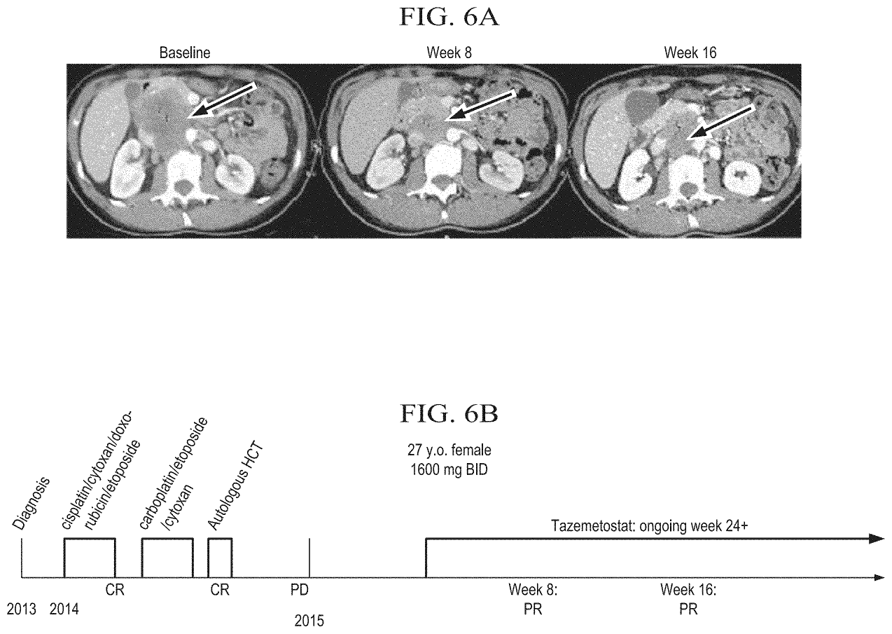

### DETAILED DESCRIPTION

INI1-negative and SMARCA4-negative tumors, such as malignant rhabdoid tumors (MRTs) and epithelioid sarcoma are serious and debilitating cancers. Approximately 1,400 patients each year in the major global markets develop these tumors, which have no established standard of care. INI1 and SMARCA4 are critical proteins of the SWI/SNF complex, which oppose the activity of EZH2. Genetic alterations or loss of function of either can result in EZH2-dependent oncogenesis in certain cancer backgrounds, thus rendering these tumors sensitive to EZH2 inhibition.

Exemplary cancers include malignant rhabdoid tumor of the ovary ((MRTO), also referred to as small cell cancer of the ovary of the hypercalcemic type (SCCOHT)).

A preferred method of treating MRTO (SCCOHT) in a subject in need thereof comprises administering to the subject a therapeutically-effective amount of tazemetostat (EPZ-6438), wherein the tazemetostat is formulated as an oral tablet, wherein the therapeutically effective amount is about 800 mg/kg, and wherein tazemetostat is administered twice per day.

EZH2 inhibitors of the disclosure are effective for treating cancers caused by a decreased abundance and/or function of a component of the SWI/SNF chromatin remodeling complex, including, for example, a decreased abundance and/or function of SMARCA4. Other components of the SWI/SNF complex that may become oncogenic markers or drivers are ARID1A, ARID2, ARID1B, SMARCB1, SMARCC1, SMARCA2, or SMARCD1. At a high level view, the SWI/SNF chromatin remodeling complex uses ATP as a source of energy for opening the chromatin to provide access for gene transcription. The activity of the multi-protein PRC2 (polycomb repressive complex 2) inhibits the opening of the chromatin, and, therefore, inhibits gene transcription. The SWI/SNF chromatin remodeling complex and the multi-protein PRC2 also interact directly with one another. However, when a function of the SWI/SNF chromatin remodeling complex is disrupted, the activity of the multi-protein PRC2 dominates, maintaining the chromatin in a closed conformation. EZH2 is the catalytic submit of PRC2. Gain-of-function mutations in EZH2 further exacerbate PRC2 dominance in cells with a disrupted SWI/SNF chromatin remodeling complex. When a function of the SWI/SNF chromatin remodeling complex is disrupted, the cell can become sensitive to EZH2-driven oncogenesis. PRC2 is the only human protein methyltransferase that can methylate the lysine (K) at position 27 within the histone protein H3 (H3K27), the only significant substrate of PRC2. PRC2 catalyzes mono-, di-, and trimethylation of H3K37 (H3K27me1, H3K27me2, and H3K27me3, respectively). H3K27me3 is an epigenetic mark for repressed gene transcription. Hyper-trimethylation of H3K27 is tumorigenic in a broad spectrum of human cancers, including, but not limited to MRT and MRTO/SCCOHT.

According to the methods of the disclosure, a “normal” cell may be used as a basis of comparison for one or more characteristics of a cancer cell, including expression and/or function of SMARCA4. As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell expresses a comparable amount of EZH2 as a cancer cell. Preferably a normal cell contains a wild type sequence for the SMARCA4 gene, expresses a SMARCA4 transcript without mutations, and expresses a SMARCA4 protein without mutations that retains all functions a normal activity levels.

As used herein, “contacting a cell” refers to a condition in which a compound or other composition of matter is in direct contact with a cell, or is close enough to induce a desired biological effect in a cell.

As used herein, “treating” or “treat” describes the management and care of a subject for the purpose of combating a disease, condition, or disorder and includes the administration of an EZH2 inhibitor of the disclosure, or a pharmaceutically acceptable salt, prodrug, metabolite, polymorph or solvate thereof, to alleviate the symptoms or complications of cancer or to eliminate the cancer.

As used herein, the term “alleviate” is meant to describe a process by which the severity of a sign or symptom of cancer is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute). Furthermore, severity describes a nuclear grade, which refers to the size and shape of the nucleus in tumor cells and the percentage of tumor cells that are dividing (see, National Cancer Institute).

In another aspect of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. Cancers may form in places where it does not cause any symptoms until the cancer has grown quite large. Ovarian cancers are considered silent killers because the cancer does not produce signs or symptoms severe enough to cause medical intervention until the tumors are either large or metastasized.

Cancer may also cause symptoms such as fever, fatigue, or weight loss. This may be because cancer cells use up much of the body's energy supply or release substances that change the body's metabolism. Or the cancer may cause the immune system to react in ways that produce these symptoms. While the signs and symptoms listed above are the more common ones seen with cancer, there are many others that are less common and are not listed here. However, all art-recognized signs and symptoms of cancer are contemplated and encompassed by the disclosure.

Treating cancer may result in a reduction in size of a tumor. A reduction in size of a tumor may also be referred to as “tumor regression”. Preferably, after treatment according to the methods of the disclosure, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment according to the methods of the disclosure, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment according to the methods of the disclosure, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

An effective amount of an EZH2 inhibitor of the disclosure, or a pharmaceutically acceptable salt, prodrug, metabolite, polymorph or solvate thereof, is not significantly cytotoxic to normal cells. For example, a therapeutically effective amount of an EZH2 inhibitor of the disclosure is not significantly cytotoxic to normal cells if administration of the EZH2 inhibitor of the disclosure in a therapeutically effective amount does not induce cell death in greater than 10% of normal cells. A therapeutically effective amount of an EZH2 inhibitor of the disclosure does not significantly affect the viability of normal cells if administration of the compound in a therapeutically effective amount does not induce cell death in greater than 10% of normal cells.

Contacting a cell with an EZH2 inhibitor of the disclosure, or a pharmaceutically acceptable salt, prodrug, metabolite, polymorph or solvate thereof, can inhibit EZH2 activity selectively in cancer cells. Administering to a subject in need thereof an EZH2 inhibitor of the disclosure, or a pharmaceutically acceptable salt, prodrug, metabolite, polymorph or solvate thereof, can inhibit EZH2 activity selectively in cancer cells.

### Malignant Rhabdoid Tumor

Malignant rhabdoid tumor (MRT) is a rare childhood tumor that occurs in soft tissues, most commonly starting in the kidneys, as well as the brain. A hallmark of certain malignant rhabdoid tumors is a loss of function of SMARCB1 (also known as INI1). INI1 is a critical component of the SWI/SNF regulatory complex, a chromatin remodeler that acts in opposition to EZH2. INI1-negative tumors have altered SWI/SNF function, resulting in aberrant and oncogenic EZH2 activity. This activity can be targeted by small molecule inhibitors of EZH2 such as tazemetostat. INI1-negative tumors are generally aggressive and are poorly served by current treatments. For example, current treatment of MRT, a well-studied INI1-negative tumor, consists of surgery, chemotherapy and radiation therapy, which are associated with limited efficacy and significant treatment-related morbidity. The annual incidence of patients with INI1-negative tumors and synovial sarcoma in major markets, including the U.S., E.U. and Japan, is approximately 2,400. Loss of function of SMARCB1/INI1 also occurs in another rare and aggressive childhood tumor, atypical teratoid rhabdoid tumor (AT/RT) of the central nervous system.

### Malignant Rhabdoid Tumor of the Ovary MRTO (Small Cell Cancer of the Ovary of the Hypercalcemic Type (SCCOHT))

MRTO/SCCOHT is an extremely rare, aggressive cancer affecting children and young women (mean age at diagnosis is 23 years). More than 65% of patients die from their disease within 2 years of diagnosis. Like MRT, these tumors are characterized by genetic loss of a SWI/SNF complex subunit, SMARCA4. SMARCA4-negative ovarian cancer cells are selectively sensitive to EZH2 inhibition with IC₅₀ values similar to those observed in MRT cells. For example, current treatment of SCCOHT consists of debulking surgery and platinum based chemotherapeutics, and shows a high rate of relapse. Differential diagnosis is broad and includes three ovarian carcinoma subtypes: granulosa cell (sex cord stromal) tumors, dysgerminoma, and high-grade serous tumors.

Standard hematoxylin and eosin (H&E) staining showed SCCOHT to be Rhabdoid-like with sheet-like arrangement of small, tightly packed, monomorphic, highly proliferative, and poorly differentiated cells whereas IHC suggests that SCCOHT is characterized by inactivation of the SMARCA4 gene leading to protein loss, and the non-mutational silencing of SMARCA2 protein. (See, e.g., Karnezis et al., J. Pathol. 2016; 238: 389-400, Jelinic et al. Nat Genet 2014, Witkowski et al., Nat. Genet. 2014; 46: 424-426, Ramos et al. Nat. Genet. 2014; 46: 427-429, Kupryjanczyk et al. Pol. J. Pathol. 2013; 64:238-246, the contents of each of which are incorporated herein by reference in their entireties). Some aspects of this disclosure provide that tumor cells and tumors, e.g., SCCOHT tumors, exhibiting SMARCA4 loss (e.g., as a result of a mutation) and SMARCA2 loss (e.g., as a result of protein loss) are sensitive to EZH2 inhibition and can thus effectively be treated with EZH2 inhibitors.

### Epithelioid Sarcoma

Epithelioid sarcoma is a rare soft tissue sarcoma, representing less than 1% of all soft tissue sarcomas. It was first clearly characterized in 1970. The most common genetic mutation found in epithelioid sarcoma is loss of INI-1 (in about 80-90%). Two variants of epithelioid sarcoma have been reported: Distal epithelioid sarcoma is associated with a better prognosis, and affects the upper and lower distal extremities (fingers, hands, forearms, or feet), while proximal epithelioid sarcoma is associated with a worse prognosis, and affects the proximal extremities (upper arm, thigh), and trunk. Epithelioid sarcoma occurs in all age groups, but is most common in young adults (median age at diagnosis is 27 years). Epithelioid sarcoma is associated with a high rate of relapse after initial treatment, and the median survival is less than 2 years when metastatic epithelioid sarcoma is diagnosed. Local recurrences and metastasis occur in about 30-50% of patients, with metastasis typically to lymph nodes, lung, bone, and brain. Treatment of epithelioid sarcoma includes surgical resection as the preferred method of treatment. For inoperable tumors or post-recurrence, conventional chemotherapy and radiation therapy, alone or in combination, are used with relatively low rates of success. About 50% of oncologists consider epithelioid sarcoma to be chemotherapy-insensitive.

### EZH2 Inhibitors

EZH2 inhibitors of the disclosure comprise, for example, tazemetostat (EPZ-6438):

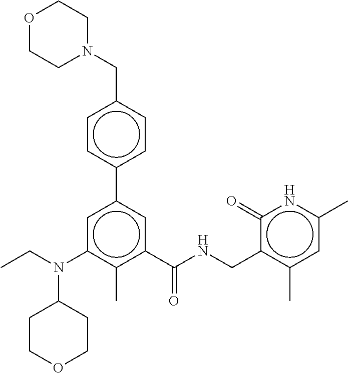

or a pharmaceutically acceptable salt thereof.

Tazemetostat is also described in U.S. Pat. Nos. 8,410,088, 8,765,732, and 9,090,562 (the contents of which are each incorporated herein in their entireties).

Tazemetostat or a pharmaceutically acceptable salt thereof, as described herein, is potent in targeting both WT and mutant EZH2. Tazemetostat is orally bioavailable and has high selectivity to EZH2 compared with other histone methyltransferases (i.e. >20,000 fold selectivity by Ki). Importantly, tazemetostat has targeted methyl mark inhibition that results in the killing of genetically defined cancer cells in vitro. Animal models have also shown sustained in vivo efficacy following inhibition of the target methyl mark. Clinical trial results described herein also demonstrate the safety and efficacy of tazemetostat.

In one embodiment, tazemetostat or a pharmaceutically acceptable salt thereof is administered to the subject at a dose of approximately 100 mg to approximately 3200 mg daily, such as about 100 mg BID to about 1600 mg BID (e.g., 100 mg BID, 200 mg BID, 400 mg BID, 800 mg BID, or 1600 mg BID), for treating a NHL. On one embodiment the dose is 800 mg BID.

EZH2 inhibitors of the disclosure may comprise, consist essentially of or consist of:

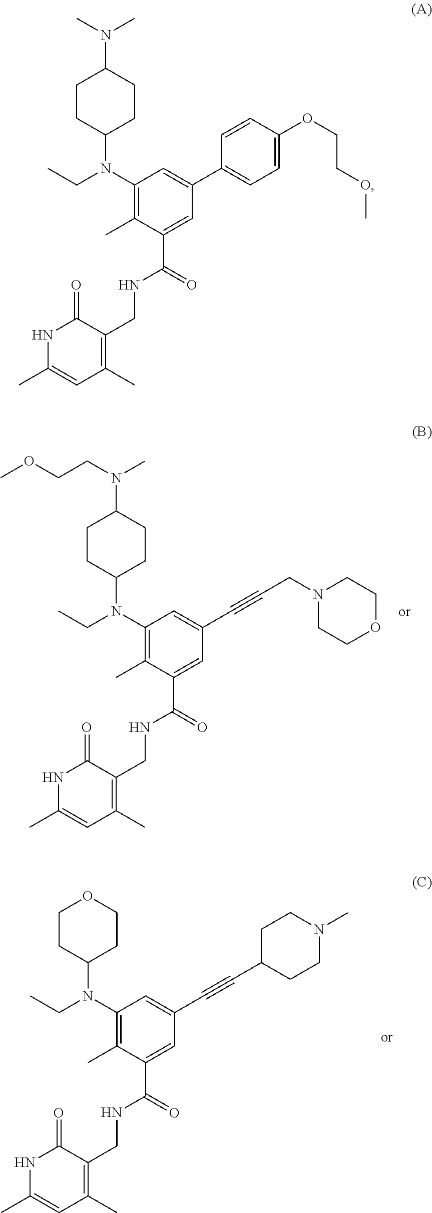

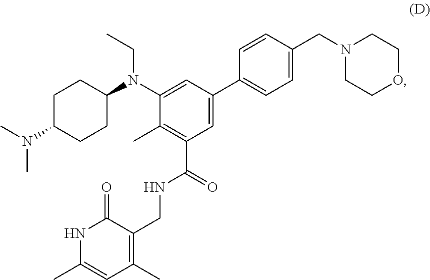 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]

or stereoisomers thereof or pharmaceutically acceptable salts and solvates thereof.

EZH2 inhibitors of the disclosure may comprise, consist essentially of or consist of Compound E:

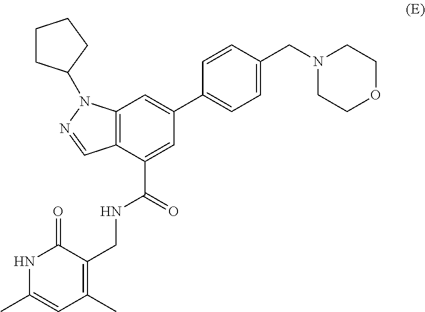 [SMILES: Cc1cc(C)c(CNC(=O)c2cc(-c3ccc(CN4CCOCC4)cc3)cc3c2cnn3C2CCCC2)c(=O)[nH]1]

or pharmaceutically acceptable salts thereof.

EZH2 inhibitors of the disclosure may comprise, consist essentially of or consist of GSK-126, having the following formula:

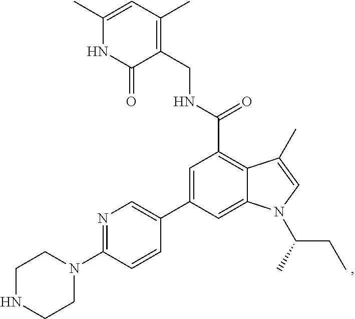 [SMILES: CC[C@H](C)n1cc(C)c2c(C(=O)NCc3c(C)cc(C)[nH]c3=O)cc(-c3ccc(N4CCNCC4)nc3)cc21]

stereoisomers thereof, or pharmaceutically acceptable salts or solvates thereof.

EZH2 inhibitors of the disclosure may comprise, consist essentially of or consist of Compound F:

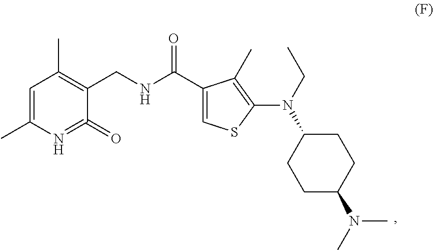

or stereoisomers thereof or pharmaceutically acceptable salts and solvates thereof.

EZH2 inhibitors of the disclosure may comprise, consist essentially of or consist of any one of Compounds Ga-Gc:

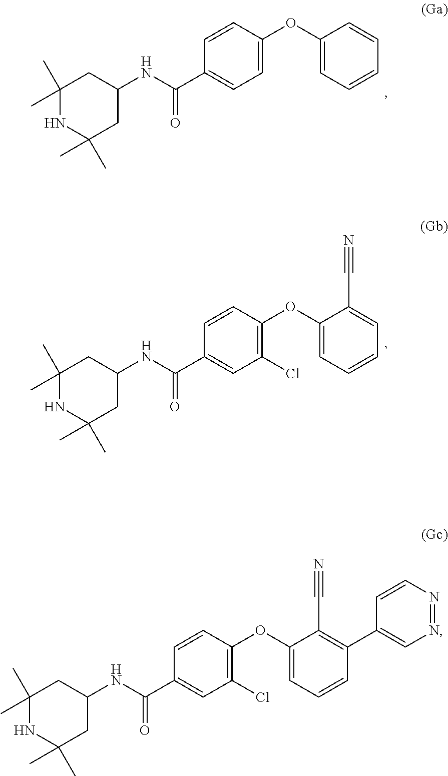 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]

or a stereoisomer, pharmaceutically acceptable salt or solvate thereof.

EZH2 inhibitors of the disclosure may comprise, consist essentially of or consist of CPI-1205 or GSK343.

Additional suitable EZH2 inhibitors will be apparent to those skilled in the art. In some embodiments of the strategies, treatment modalities, methods, combinations, and compositions provided herein, the EZH2 inhibitor is an EZH2 inhibitor described in U.S. Pat. No. 8,536,179 (describing GSK-126 among other compounds and corresponding to WO 2011/140324), the entire contents of each of which are incorporated herein by reference.

In some embodiments of the strategies, treatment modalities, methods, combinations, and compositions provided herein, the EZH2 inhibitor is an EZH2 inhibitor described in PCT/US2014/015706, published as WO 2014/124418, in PCT/US2013/025639, published as WO 2013/120104, and in U.S. Ser. No. 14/839,273, published as US 2015/0368229, the entire contents of each of which are incorporated herein by reference.

In one embodiment, the compound disclosed herein is the compound itself, i.e., the free base or “naked” molecule. In another embodiment, the compound is a salt thereof, e.g., a mono-HCl or tri-HCl salt, mono-HBr or tri-HBr salt of the naked molecule.

Compounds disclosed herein that contain nitrogens can be converted to N-oxides by treatment with an oxidizing agent (e.g., 3-chloroperoxybenzoic acid (mCPBA) and/or hydrogen peroxides) to afford other compounds suitable for any methods disclosed herein. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds disclosed herein can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds disclosed herein may be depicted as different chiral isomers or geometric isomers. It should also be understood that when compounds have chiral isomeric or geometric isomeric forms, all isomeric forms are intended to be included in the scope of the disclosure, and the naming of the compounds does not exclude any isomeric forms.

Furthermore, the structures and other compounds discussed in this disclosure include all atropic isomers thereof “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerization is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of keto-enol equilibria is between pyridin-2(1H)-ones and the corresponding pyridin-2-ols, as shown below.

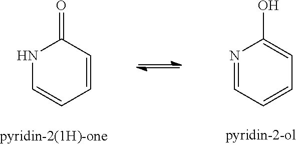 [SMILES: O=c1cccc[nH]1 ; Oc1ccccn1]

It is to be understood that the compounds disclosed herein may be depicted as different tautomers. It should also be understood that when compounds have tautomeric forms, all tautomeric forms are intended to be included in the scope of the disclosure, and the naming of the compounds does not exclude any tautomer form.

The compounds disclosed herein include the compounds themselves, as well as their salts and their solvates, if applicable. A salt, for example, can be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms. In the salt form, it is understood that the ratio of the compound to the cation or anion of the salt can be 1:1, or any ration other than 1:1, e.g., 3:1, 2:1, 1:2, or 1:3.

Additionally, the compounds disclosed herein, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein. For example, all of the compounds represented by Formula (I) are aryl- or heteroaryl-substituted benzene compounds, and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

### Pharmaceutical Formulations

The present disclosure also provides pharmaceutical compositions comprising at least one EZH2 inhibitor described herein in combination with at least one pharmaceutically acceptable excipient or carrier.

A “pharmaceutical composition” is a formulation containing the EZH2 inhibitors of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the disclosure includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the disclosure can be administered to a subject in many of the well-known methods currently used for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of an EZH2 inhibitor, composition, or pharmaceutical composition thereof effective to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer, including but not limited to, malignant rhabdoid tumor (MRT), MRT of the ovary (MRTO) and small cell cancer of the ovary of the hypercalcemic type (SCCOHT).

For any EZH2 inhibitor of the disclosure, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing an EZH2 inhibitor of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds (i.e. EZH2 inhibitors of the disclosure) can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The compounds of the present disclosure are capable of further forming salts. All of these forms are also contemplated within the scope of the claimed disclosure.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The EZH2 inhibitors of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The EZH2 inhibitors of the present disclosure can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl)N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p 1-92, Elesevier, New York-Oxford (1985).

The EZH2 inhibitors, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

The dosage regimen can be daily administration (e.g. every 24 hours) of a compound of the present disclosure. The dosage regimen can be daily administration for consecutive days, for example, at least two, at least three, at least four, at least five, at least six or at least seven consecutive days. Dosing can be more than one time daily, for example, twice, three times or four times daily (per a 24 hour period). The dosing regimen can be a daily administration followed by at least one day, at least two days, at least three days, at least four days, at least five days, or at least six days, without administration.

Techniques for formulation and administration of the disclosed compounds of the disclosure can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

Methods of the disclosure for treating cancer including treating a malignant rhabdoid tumor (MRT). In preferred embodiments, methods of the disclosure are used to treat a subject having a malignant rhabdoid tumor of the ovary (MRTO). MRTO may also be referred to as small cell cancer of the ovary of the hypercalcemic type (SCCOHT). In certain embodiments, the MRTO or SCCOHT and/or the subject are characterized as SMARCA4-negative. As used herein SMARCA4-negative cells contain a mutation in the SMARCA4 gene, corresponding SMARCA4 transcript (or cDNA copy thereof), or SMARCA4 protein that prevents transcription of a SMARCA4 gene, translation of a SMARCA4 transcript, and/or decreases/inhibits an activity of a SMARCA4 protein. The SMARCA4-negative status of a cell renders that cell sensitive to EZH2 driven oncogenesis.

Methods of the disclosure may be used to treat a subject who is SMARCA4-negative or who has one or more cells that may be SMARCA4-negative. SMARCA4 expression and/or SMARCA4 function may be evaluated by fluorescent and non-fluorescent immunohistochemistry (IHC) methods, including well known to one of ordinary skill in the art. In a certain embodiment the method comprises: (a) obtaining a biological sample from the subject; (b) contacting the biological sample or a portion thereof with an antibody that specifically binds SMARCA4; and (c) detecting an amount of the antibody that is bound to SMARCA4. Alternatively, or in addition, SMARCA4 expression and/or SMARCA4 function may be evaluated by a method comprising: (a) obtaining a biological sample from the subject; (b) sequencing at least one DNA sequence encoding a SMARCA4 protein from the biological sample or a portion thereof; and (c) determining if the at least one DNA sequence encoding a SMARCA4 protein contains a mutation affecting the expression and/or function of the SMARCA4 protein. SMARCA4 expression or a function of SMARCA4 may be evaluated by detecting an amount of the antibody that is bound to SMARCA4 and by sequencing at least one DNA sequence encoding a SMARCA4 protein, optionally, using the same biological sample from the subject.

All percentages and ratios used herein, unless otherwise indicated, are by weight.

Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### EXAMPLES

In order that the invention disclosed herein may be more efficiently understood, examples are provided below. It should be understood that these examples are for illustrative purposes only and are not to be construed as limiting the disclosure in any manner.

### Example 1: Treatment of SMARCA4-Negative MRTO/SCCOHT with Tazemetostat

A human female of 27 years of age diagnosed with SMARCA4-negative MRTO/SCCOHT was successfully treated with 1600 mg of EPIZ-6438 (Tazemetostat) administered twice daily (BID) by oral tablet. Tumor size decreased from baseline after 8 weeks of treatment and, further decreased from the 8 week measurement after 16 weeks of treatment.

The subject had been diagnosed with SMARCA4-negative MRTO/SCCOHT in 2013. Throughout 2014, the subject was treated with a course of cisplatin/cytoxan/doxorubicin/etoposide and, subsequently, a course of carboplatin/etoposide/cytoxan. Neither course of treatment was successful. The subject then received an autologous hematopoietic cell transplantation that also failed to treat the SMARCA4-negative MRTO/SCCOHT.

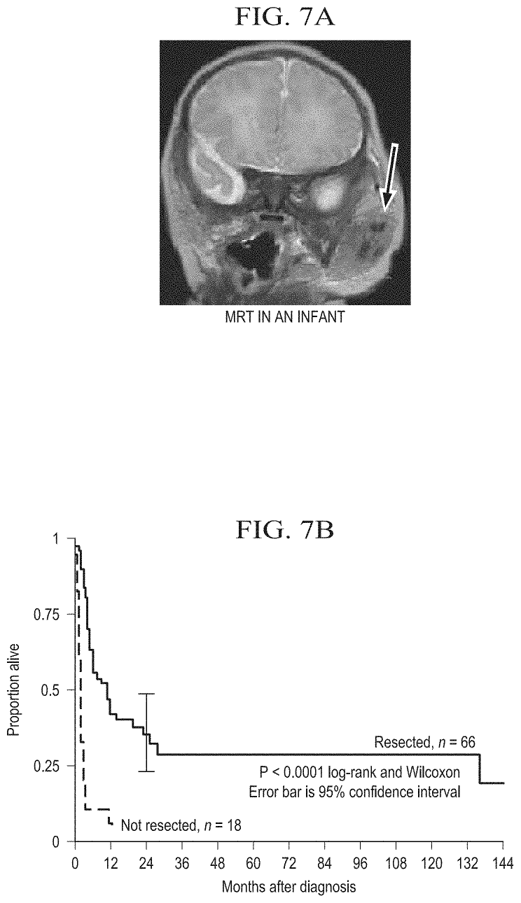

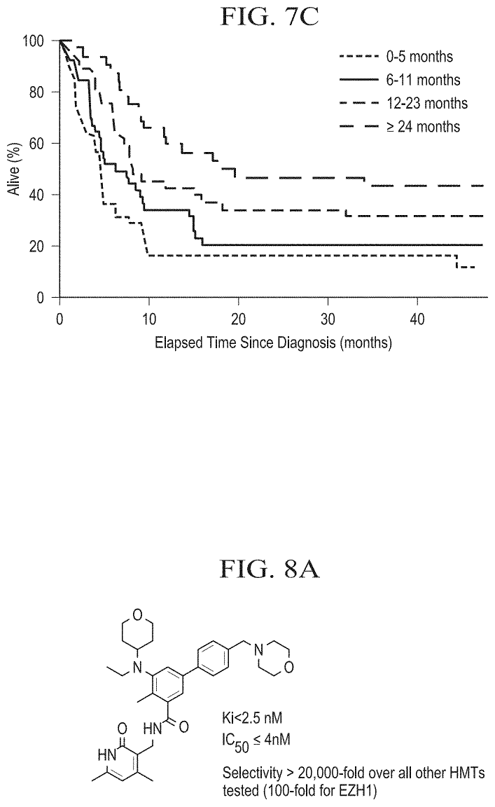

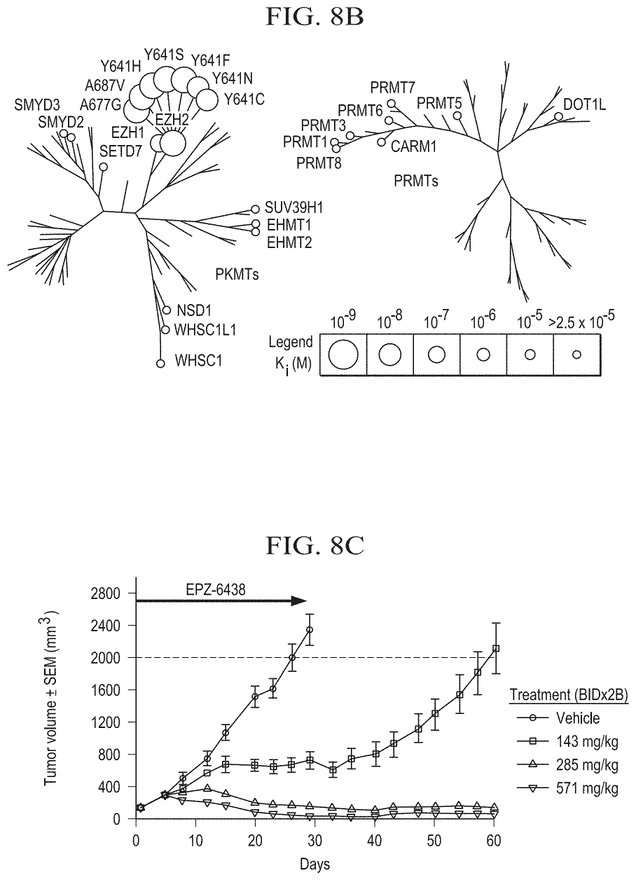

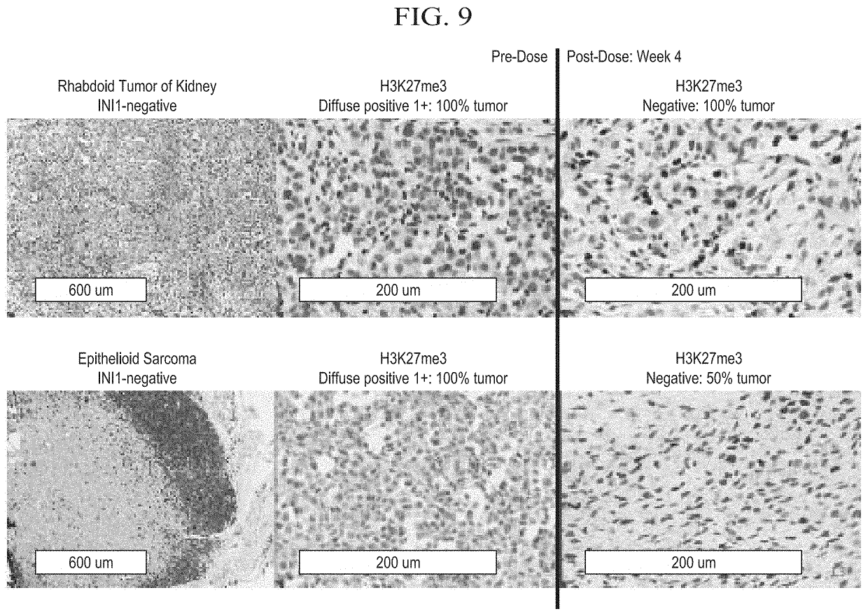

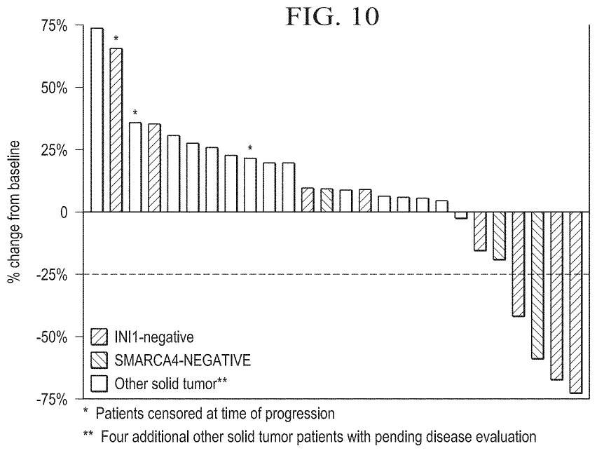

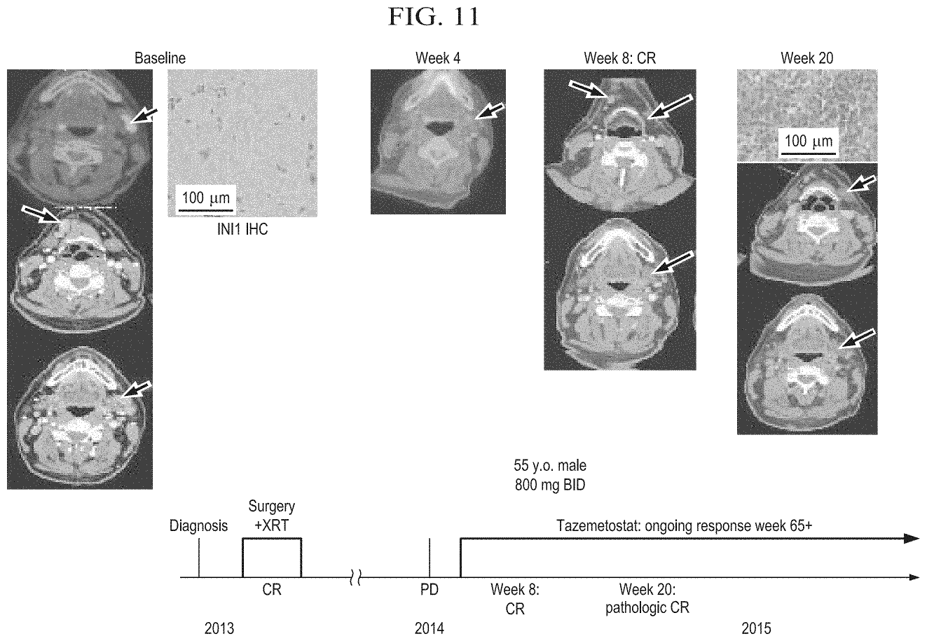

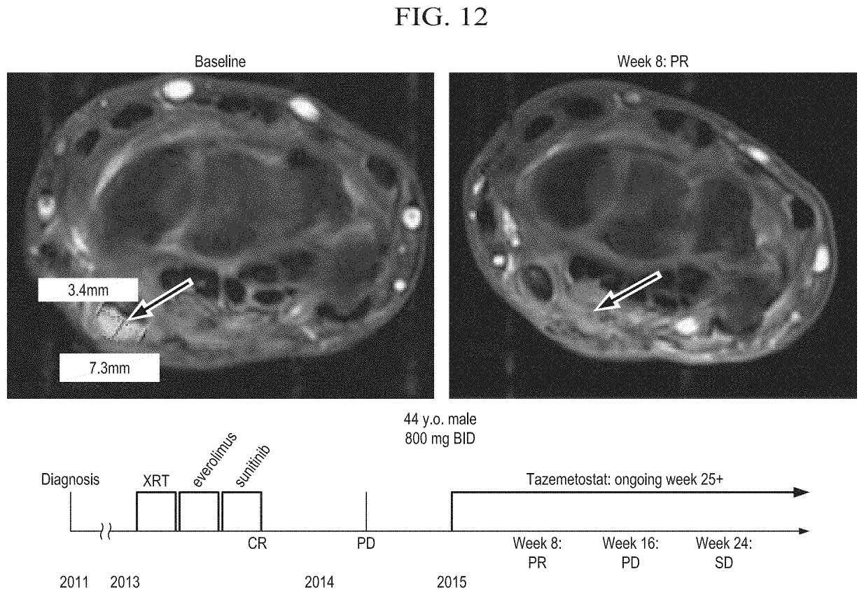

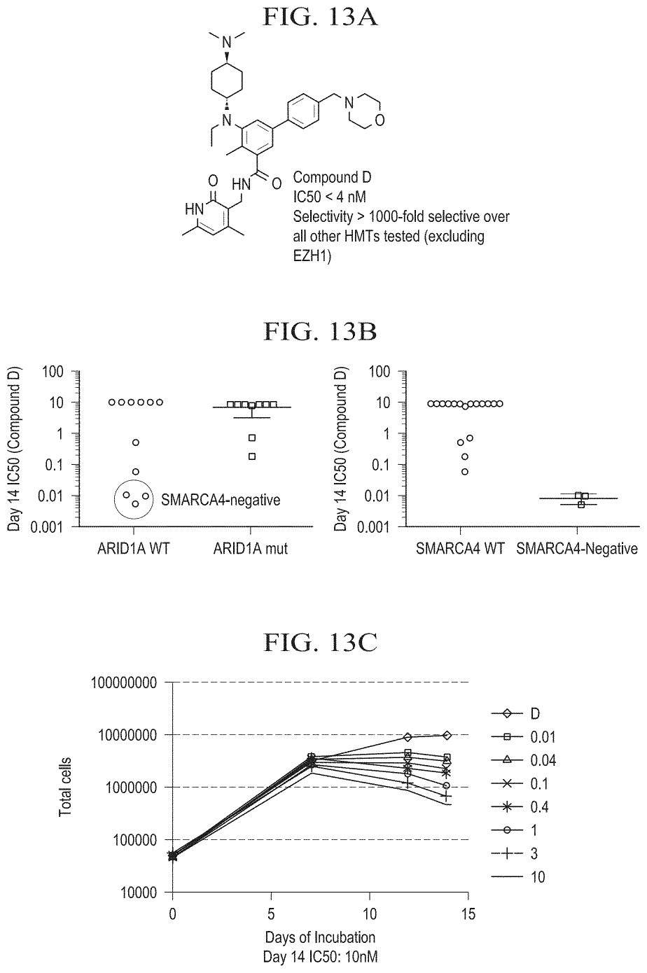

The subject is currently undergoing therapy with 1600 mg of tazemetostat administered twice daily (BID) by oral tablet. Preliminary results are provided in FIG. 6A, however, the treatment is ongoing and will continue through at least week 24.

### Example 2: Remission for INI1- and SMARCA4-Negative Tumors

Treatment of INI1- and SMARCA4-negative tumors with tazemetostat induces pharmacodynamics inhibition of HeK27me3 in tumor tissue.

Assessments of clinical activity of MRT and MRTO/SCCOHT following tazemetostat treatment show stable disease for at least six months, partial remission or complete remission.

### Example 3: Whole Exome Sequencing Identifying Variants in SWI/SNF Subunits

Archive or baseline formalin-fixed paraffin-embedded (FFPE) samples were submitted for genomic DNA isolation (n=25). 18 of the 25 samples had enough DNA to proceed into library preparation and whole exome sequencing. 16 of the 18 samples passed sequencing quality control. Greater than 300× median sequencing coverage of SWI/SNF components. Variants identified in dbSNP and those with <5% allelic frequency were filtered out.

Genetic variants of SWI/SNF complex characterized in phase I solid tumor patients (see Table 1). SMARCA4 nonsense mutation detected in a patient achieving partial remission (PR). Nonsense and frame shift mutations of SMARCB1 identified in patients exhibiting INI1 protein loss through immunohistochemistry (IHC). Additional somatic mutations identified in SWI/SNF components in non-responding patients only, e.g. 3/13 patients with ARID1A mutations.

TABLE 1

| Response | Patients with | Gene |
|---|---|---|
| Category (n) | variant detected | Variant (n) |
| SD ≥ 6 mo, | 2 | SMARCB1 (1) |
| PR or CR (4) | | SMARCA4 (1) |
| SD < 6 mo or | 6 | ARID1A (3) |
| PD (12) | | SMARCB1 (3) |
| | | SMARCC1 (2) |
| | | ARID2 (1) |
| | | ARID1B (1) |
| | | SMARCA2 (1) |
| | | SMARCA4 (1) |
| | | SMARCD1 (1) |

Table 2 describes a Phase 1 clinical trial design (sponsor protocol no.: E7438-G000-001, ClinicalTrials.gov identifier: NCT01897571). The study population included subjects with relapsed or refractory solid tumors or B-cell lymphoma. Subjects received a 3+3 dose-escalation in expansion cohorts receiving 800 mg BID and 1600 mg BID, respectively, or a cohort for ascertaining the effect of food on dosing at 400 mg BID. The primary endpoint was a determination of recommended phase II dose (RP2D)/maximum tolerated dose (MTD). Secondary endpoints included safety, pharmacokinetics (PK), pharmacodynamics (PD) and tumor response, assessed every 8 wks.

TABLE 2

| Dose | Patients | Solid tumors | B-cell NHL |
|---|---|---|---|
| (mg BID) | (n = 51) | (n = 30) | (n = 21) |
| 100* | 6 | 5 | 1 |
| 200 | 3 | 1 | 2 |
| 400 | 3 | 2 | 1 |
| 800 | 14 | 6 | 8 |
| 1600 | 12 | 8 | 4 |
| Food Effect | 13 | 8 | 5 |

*2 formulations

Table 3 illustrates the different patient tumor types.

TABLE 3

| | |
|---|---|
| Relapsed or refractory solid tumor | N = 30 |

| | | |
|---|---|---|
| INI1-negative | Malignant rhabdoid tumor | 5 |
| (SMARCB1)* | Epithelioid sarcoma | 3 |
| SMARCA4-negative* | Malignant rhabdoid tumor of | 2 |
| | ovary (SCCOHT) | |
| | Thoracic sarcoma | 1 |

| | |
|---|---|
| Synovial sarcoma | 3 |
| GI malignancy | 9 |
| GU malignancy | 2 |
| GYN malignancy (non-SCCOHT) | 2 |
| CNS tumor/other sarcoma | 3 |
| Relapsed or refractory NHL | N = 21 |

*INI1- or SMARCA4-negative by IHC

Table 4 summarizes solid tumor patient demographics.

TABLE 4

| | Characteristic | | N = 30 (%) |
|---|---|---|---|
| Median age, years [range] | | | 53.5 [18-79] |
| Sex (M/F) | | | 12/18 |
| | # of prior | 0* | 3 (10) |
| | therapeutic | 1 | 5 (17) |
| | systemic | 2 | 6 (20) |
| | regimens | 3 | 4 (13) |
| | | 4 | 2 (7) |
| | | ≥5 | 10 (33) |

| | |
|---|---|
| Prior autologous hematopoietic cell transplant | 1 (3) |
| Prior radiotherapy | 17 (57) |

*Malignant rhabdoid tumor pts - definitive surgery and/or adjuvant radiotherapy only

Table 5 describes a safety profile in NHL (non-Hodgkin's lymphoma) and solid tumor patients (n-51).

TABLE 5

| | All Events* | | All Treatment-Related | |
|---|---|---|---|---|
| | All Grades | Grade ≥3 | All Grades | Grade ≥3 |
| Asthenia | 28 | 1 | 12 | 0 |
| Decreased | 11 | 3 | 3 | 1 |
| appetite | | | | |
| Thrombocytopenia | 10 | 2 | 6 | 1 |
| Nausea | 8 | 0 | 7 | 0 |
| Dyspnea | 8 | 0 | 0 | 0 |
| Anemia | 7 | 1 | 3 | 0 |
| Constipation | 7 | 0 | 2 | 0 |
| Vomiting | 6 | 0 | 3 | 0 |
| Dysgeusia | 5 | 0 | 5 | 0 |
| Muscle Spasms | 5 | 0 | 2 | 0 |
| Hypertension | 3 | 1 | 1 | 1 |
| Elevated LFTs | 2 | 1 | 0 | 1 |
| Neutropenia | 2 | 1 | 1 | 1 |

*Frequency ≥10% regardless of attribution

Table 6 illustrates clinical activity in patients with INI1- or SMARCA4-negative tumors.

TABLE 6

| | | Dose | | Time on study |
|---|---|---|---|---|
| Tumor | | (mg BID) | Best Response | (weeks) |
| INI1-negative | Malignant | 800 | CR week 8* | 65+ |
| | rhabdoid tumor | 1600 | PR week 8 | 16 |
| | | 1600 | SD week 8 | 17 |
| | | 400 | SD week 8 | 12+ |
| | | 800 | PD week 8 | 35 |
| | Epithelioid | 800 | PR week 8 | 25+ |
| | sarcoma | 800 | SD week 8 | 24+ |
| | | 400 | PD week 8 | 11 |
| SMARCA4-negative | Malignant | 1600 | PR week 8* | 25+ |
| | rhabdoid tumor of | 1600 | SD week 8 | 26+ |
| | ovary (SCCOHT) | | | |
| | Thoracic sarcoma | 1600 | PD week 5 | 6 |

*Confirmed response by RECIST 1.1 criteria
+/Patients who remain on study

### Example 4: Preclinical and Clinical Evaluation of EZH2 Inhibitors in Models of Small Cell Carcinoma of the Ovary, Hypercalcemic Type (SCCOHT)

The H3K27 histone methyltransferase EZH2 is the catalytic component of the polycomb repressive complex 2 (PRC2), and is amplified, overexpressed, or mutated in multiple cancer types, supporting its function as an oncogene. In addition to genetic alterations in EZH2 itself, distal genetic changes in other proteins can lead to oncogenic dependency on EZH2 activity. It has been established that cell lines and xenografts deficient in INI1 (SNF5/SMARCB1), a core component of the SWItch/Sucrose Non-Fermentable (SWI/SNF) chromatin remodeling complex, display profound sensitivity and durable regressions in the presence of the selective EZH2 inhibitor tazemetostat (EPZ-6438, see, e.g., Knutson et al. PNAS 2013; 110:7922-7927, which is incorporated herein by reference in its entirety).

Following the preclinical observation of activity in lymphoma and INI1-negative tumors, a Phase 1 dose escalation study of tazemetostat was initiated (ClinicalTrials.gov identifier: NCT01897571). As reported, a complete response was observed in a patient with an INI1-negative (confirmed by IHC) relapsed malignant rhabdoid tumor. It has been suggested that rhabdoid tumors are addicted to or dependent from dysregulated PRC2 activity. The previously proposed antagonistic relationship of SWI/SNF with PRC2, which is perturbed in INI1-deficient tumors has been confirmed. The loss of INI1 induces inappropriate SWI/SNF function, abrogating the repression of PRC2 activity. This results in polycomb target genes, such as those involved in differentiation and tumor suppression, to become aberrantly repressed. In addition to deletion of INI1, there are numerous reports describing genetic alterations in other SWI/SNF complex members. Given the oncogenic dependency of INI1-deficient tumors on PRC2 activity, the sensitivity of other SWI/SNF mutated cancer types to EZH2 inhibition was investigated in this study. Specifically, the effects of EZH2 inhibition in ovarian cancers carrying somatic mutations in the SWI/SNF complex members ARID1A and SMARCA4 were investigated in the study.

A panel of ovarian cancer cell lines of different histologies was subjected to proliferation assays in 2-D tissue culture for 14 days in the presence of increasing concentrations of an EZH2 inhibitor. Selected cell lines were also tested in 3-D cultures. It was found that ovarian cancer cell lines deficient in the SWI/SNF components SMARCA2 and SMARCA4 (also known as BRG1) are among the most sensitive to EZH2 inhibition, as demonstrated by decreased proliferation and/or morphology changes, at concentrations that are clinically achievable. In contrast, mutations in ARID1A, another SWI/SNF component, were not observed to broadly confer sensitivity to EZH2 inhibition in ovarian cancer cell lines in either 2-D or 3-D in vitro assays. Clinical activity was observed in a Phase 1 trial in two patients with SCCOHT (SMARCA4-negative) treated with tazemetostat.

SCCOHT is characterized by SMARCA2 and SMARCA4 loss and shows a dependency on EZH2, demonstrated in preclinical and clinical studies. In particular, the three SCCOHT cell lines tested were the most sensitive to Compound D in 14-day proliferation assays (IC₅₀: 5-17 nM) out of −20 ovarian cell lines tested. Clinical activity (SD≥6 months and confirmed PR) was observed in patients with relapsed SMARCA4-negative malignant rhabdoid tumor of ovary (SCCOHT).

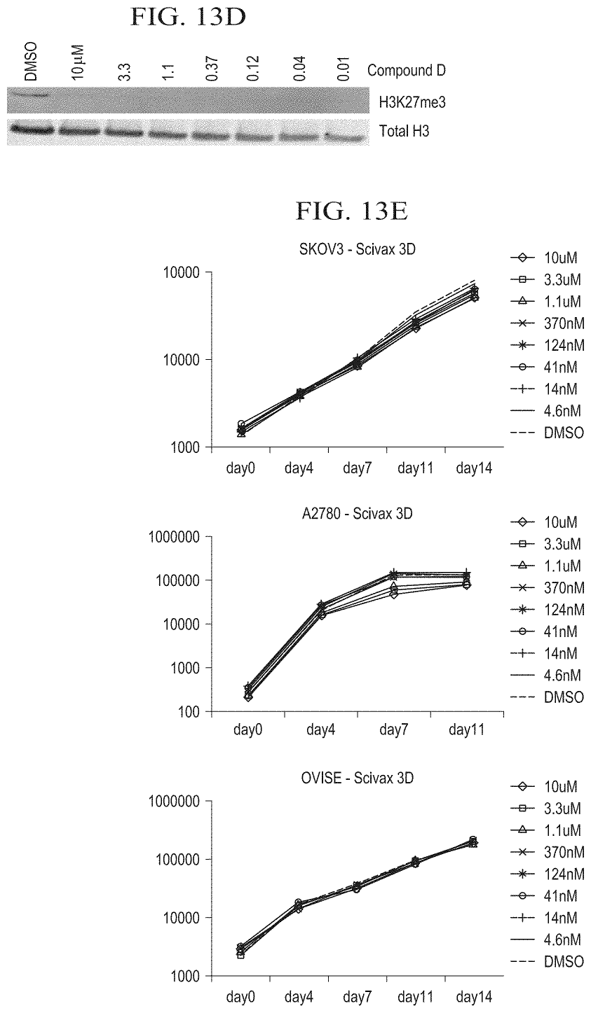

Examination of SMARCA2/4 protein levels across ovarian cancer cell lines led to the identification of two additional, previously misclassified, SCCOHT cell lines (FIG. 14).

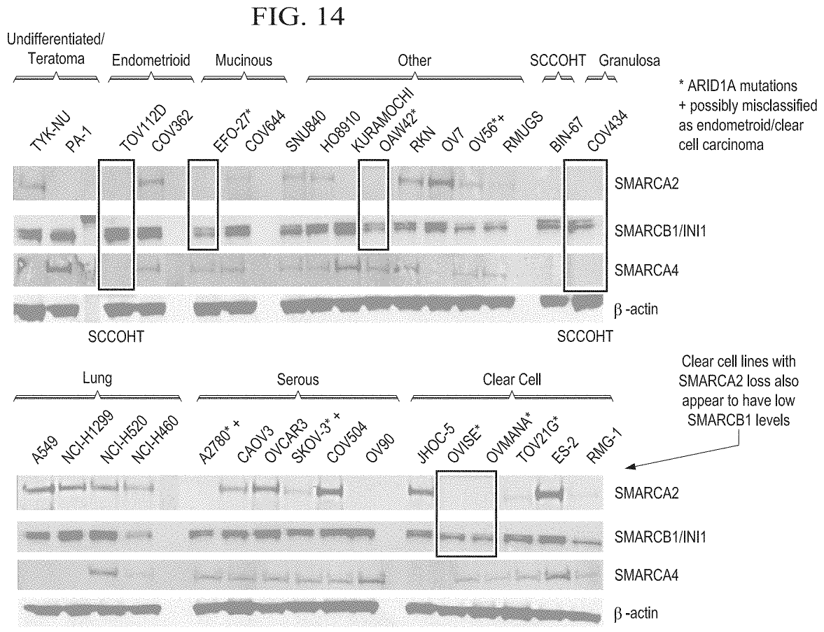

An immunohistochemical analysis of core SWI/SNF proteins in SCCOHT cell lines showed dual loss of SMARCA4/BRG1 and SMARCA2/BRM (FIG. 15).

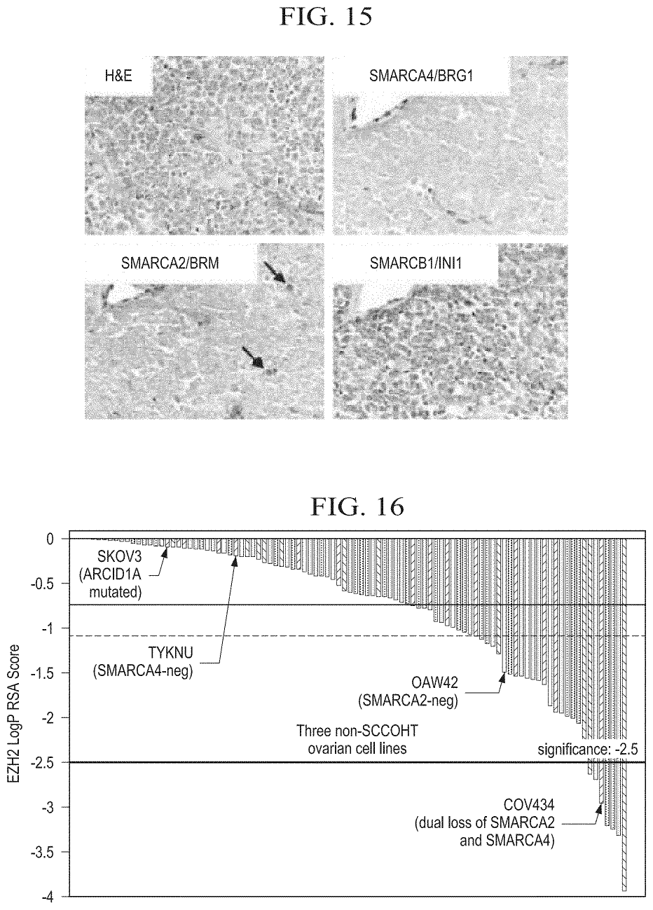

A SCCOHT cell line (COV434) tested in a CRISPR pooled screen was sensitive to EZH2 knockout while the other three ovarian cell lines were not as sensitive (FIG. 16).

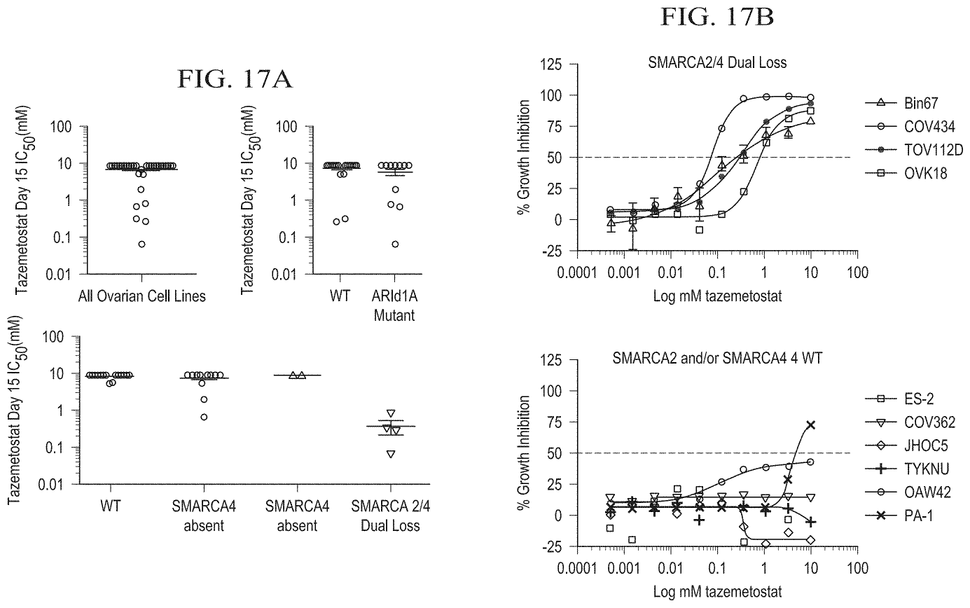

Dual SMARCA2 and SMARCA4 deficient ovarian cell lines were found to be most sensitive to tazemetostat in long-term proliferation assays (FIG. 17A). Thirty-three ovarian cell lines were tested in long-term proliferation assays with tazemetostat. IC₅₀s between 0.073 μM and >10 μM were observed. Cell lines with loss of both SMARCA2 and SMARCA4 were most sensitive to tazemetostat (IC₅₀ values of less than 1 μM).

Dose-dependent inhibition of cell growth was observed upon tazemetostat treatment in four SMARCA2-deficient and SMARCA4-deficient cell lines. Lower sensitivity was observed in single-deficient or WT cell lines (SMARCA4-deficient JHOC-5 and TYKNU; SMARCA2-deficient PA-1 and OAW42; or SMARCA2 and SMARCA4 WT ES-2 or COV362 cell lines, FIG. 17B).

Sensitivity to EZH2 inhibition was examined in various cancer cell lines with similar mutations or loss of SWI/SNF components. Table 7 summarizes the EZH2 activity in additional SWI/SNF altered cancers, including lung adenocarcinoma.

TABLE 7

| | | | | Compound D |
|---|---|---|---|---|
| Cell line | SMARCA2 | SMARCA4 | KRAS | IC₅₀ [M] |
| A427 | mRNA/protein loss | Mutations | Mutations | 2.3 |
| NCl-H23 | mRNA/protein loss | Mutations | Mutations | 10 |
| NCl-H522 | mRNA/protein loss | Mutations | No mutations or | 1.14 |
| | | | info not available | |
| A549 | No mutations or | Mutations | Mutations | 10* |
| | info not available | | | |
| NCl-H1299 | No mutations or | Mutations | No mutations or | 10 |
| | info not available | | info not available | |
| NClH838 | No mutations or | Mutations | No mutations or | 10 |
| | info not available | | info not available | |
| NClH1793 | No mutations or | Mutations | No mutations or | 10 |
| | info not available | | info not available | |
| Calu-6 | No mutations or | No mutations or | Mutations | 0.001 |
| | info not available | info not available | | |
| NCl-H441 | No mutations or | No mutations or | Mutations | 10* |
| | info not available | info not available | | |
| H460 | No mutations or | No mutations or | Mutations | 10* |
| | info not available | info not available | | |
| NClH2122 | No mutations or | No mutations or | Mutations | 10 |
| | info not available | info not available | | |
| NClH1734 | No mutations or | No mutations or | Mutations | 10 |
| | info not available | info not available | | |
| NClH1373 | No mutations or | No mutations or | Mutations | 10 |
| | info not available | info not available | | |
| NClH1993 | No mutations or | No mutations or | No mutations or | 10 |
| | info not available | info not available | info not available | |
| NClH2110 | No mutations or | No mutations or | No mutations or | 10 |
| | info not available | info not available | info not available | |
| Calu-3 | No mutations or | No mutations or | No mutations or | 10 |
| | info not available | info not available | info not available | |
| NClH1563 | No mutations or | No mutations or | No mutations or | 10 |
| | info not available | info not available | info not available | |
| HCC827 | No mutations or | No mutations or | No mutations or | 10* |
| | info not available | info not available | info not available | |

*Proquinase 3D IC₅₀

### Example 5: In Vivo Treatment of Tumors in a SCCOHT Xenograft Model (Bin-67)

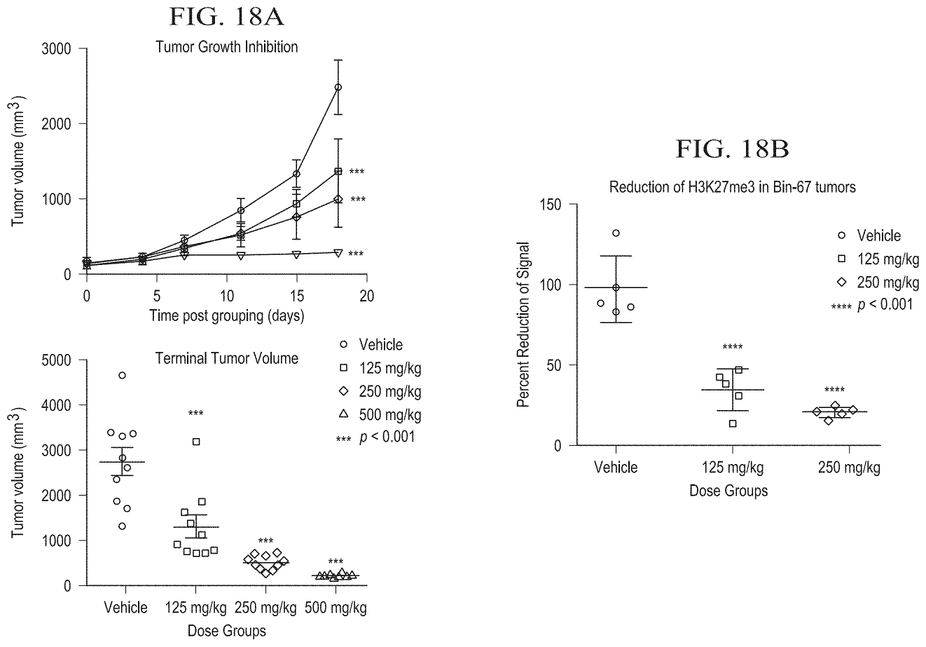

In vivo xenograft tumors from SCCOHT cell line Bin-67 were dosed with tazemetostat for 18 days. Tumors showed statistically significant differences in volume compared to vehicle after 18 days in the Bin-67 model (FIG. 18A). EZH2 target inhibition was assessed by H3K27me3 levels in xenograft tissue collected on day 18 (FIG. 18B). Each point represents the ratio of H3K27me3 to total H3 from the tumor of a single animal.

### Example 6: In Vivo Treatment of Tumors in a SCCOHT Xenograft Model (COV434)

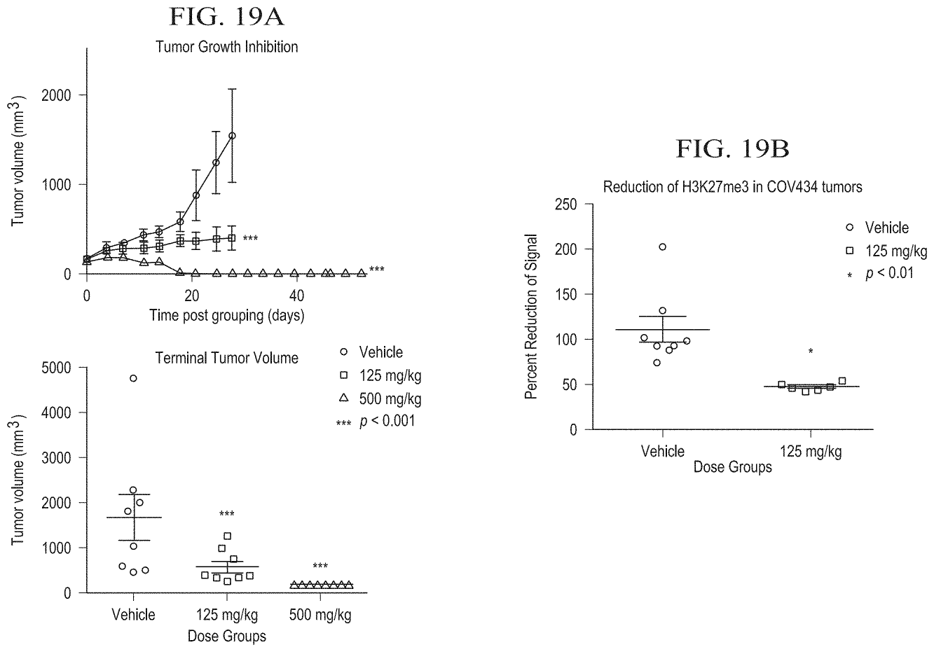

In vivo xenograft tumors from SCCOHT cell line COV434 were dosed with tazemetostat for 28 days. Tumors showed statistically significant differences in volume compared to vehicle after 28 days in the COV434 model (FIG. 19A). After day 28, a portion of the COV434 xenograft cohort were retained to monitor for tumor regrowth while under no treatment, of which there was none. EZH2 target inhibition was assessed by H3K27me3 levels in xenograft tissue collected on day 28 (FIG. 19B). Each point represents the ratio of H3K27me3 to total H3 from the tumor of a single animal.

### Example 7: In Vivo Treatment of Tumors in a SCCOHT Xenograft Model (TOV112D)

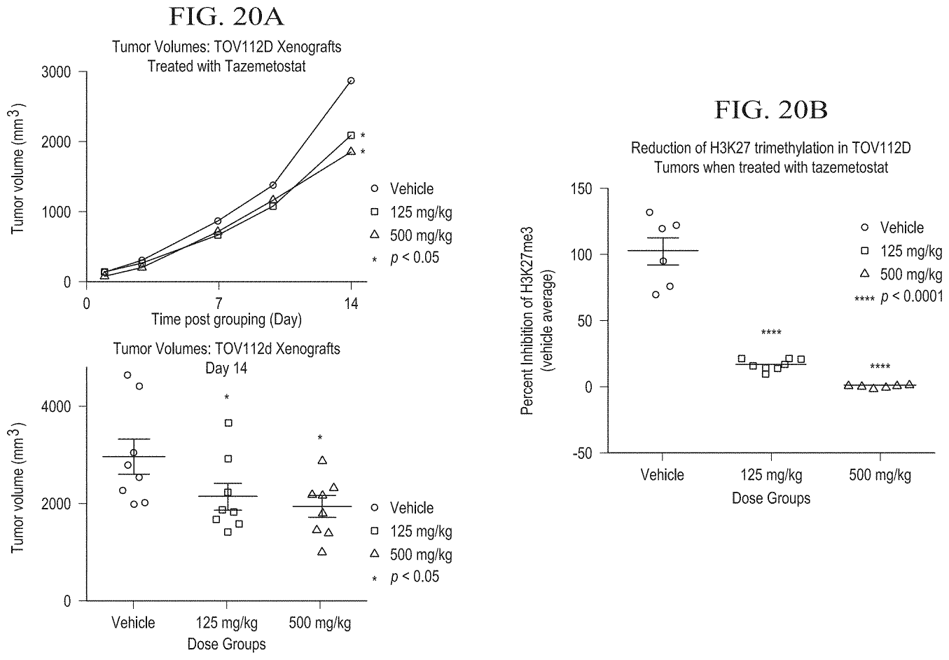

In vivo xenograft tumors from SCCOHT line TOV112D were dosed with tazemetostat for 14 days, twice daily. Tumors showed statistically significant differences in volume compared to vehicle after 14 days in the TOV112D model (FIG. 20A). EZH2 target inhibition was measured by H3K27me3 levels in xenograft tissue collected on day 14 (FIG. 20B). Each point represents the ratio of H3K27me3 to total H3 from the tumor of a single animal.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow. Where names of cell lines or genes are used, abbreviations and names conform to the nomenclature of the American Type Culture Collection (ATCC) or the National Center for Biotechnology Information (NCBI), unless otherwise noted or evident from the context.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of treating a malignant rhabdoid tumor in a subject in need thereof comprising administering to the subject a therapeutically effective amount of an enhancer of a zeste homolog 2 (EZH2) inhibitor, optionally, wherein the MRT is INI1-negative, INI1-deficient or epithelioid sarcoma.

2. A method of treating malignant rhabdoid tumor of the ovary (MRTO)/small cell cancer of the ovary of the hypercalcemic type (SCCOHT) in a subject in need thereof comprising administering to the subject a therapeutically effective amount of an EZH2 inhibitor.

3. The method of claim 1, wherein the EZH2 inhibitor inhibits tri-methylation of lysine 27 of histone 3 (H3K27).

4. The method of claim 1, wherein the EZH2 inhibitor is 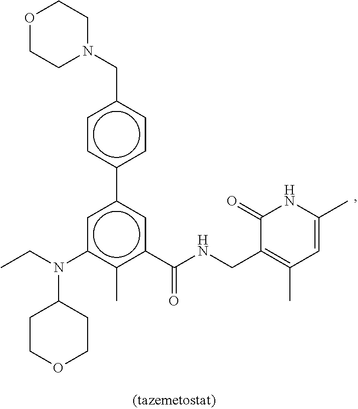
or a pharmaceutically acceptable salt thereof.

5. The method of claim 1, wherein the EZH2 inhibitor is 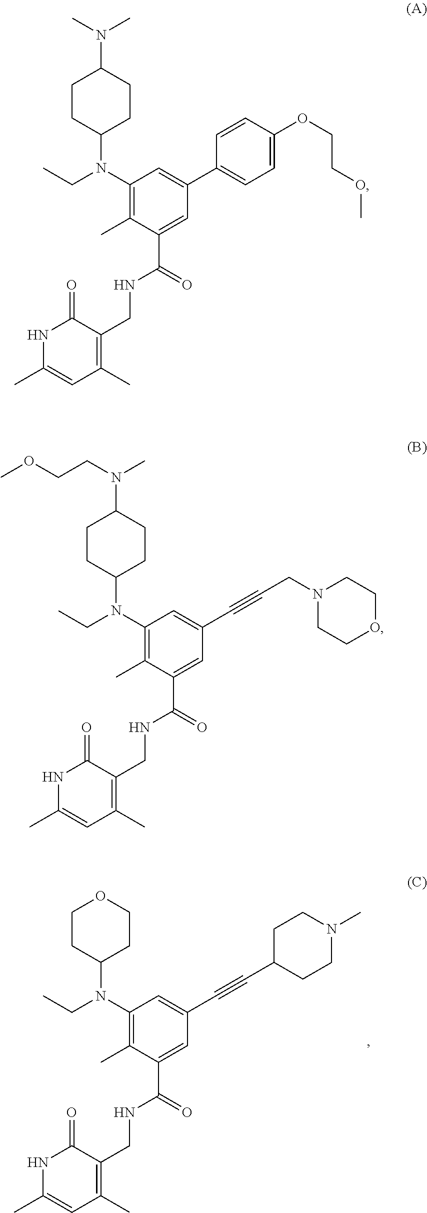 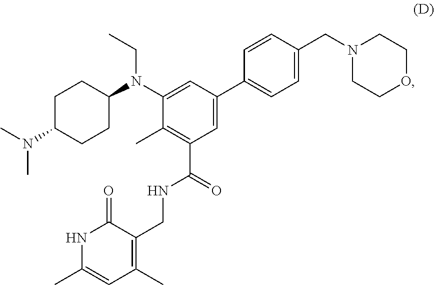 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]
a stereoisomer, a pharmaceutically acceptable salt and/or a solvate thereof.

6. The method of claim 1, wherein the EZH2 inhibitor is 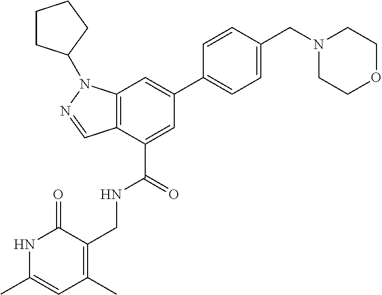 [SMILES: Cc1cc(C)c(CNC(=O)c2cc(-c3ccc(CN4CCOCC4)cc3)cc3c2cnn3C2CCCC2)c(=O)[nH]1]
or a pharmaceutically acceptable salt thereof.

7.-9. (canceled)

10. The method of claim 1, wherein the EZH2 inhibitor is administered orally.

11. The method of claim 1, wherein the EZH2 inhibitor is formulated as an oral tablet.

12. The method of claim 1, wherein the EZH2 inhibitor is administered at a dose of between 10 mg/kg/day and 1600 mg/kg/day.

13. The method of claim 1, wherein the EZH2 inhibitor is administered at a dose of about 100 mg, 200 mg, 400 mg, 800 mg, or 1600 mg.

14. The method of claim 13, wherein the EZH2 inhibitor is administered at a dose of about 800 mg.

15. The method of claim 1, wherein the EZH2 inhibitor is administered twice per day (BID).

16. (canceled)

17. The method of claim 1, wherein the tumor is SMARCA4-negative.

18. The method of claim 17, wherein SMARCA4 expression or a function of SMARCA4 is evaluated by a method comprising:
(a) obtaining a biological sample from the subject;
(b) contacting the biological sample or a portion thereof with an antibody that specifically binds SMARCA4; and
(c) detecting an amount of the antibody that is bound to SMARCA4.

19. The method of claim 17, wherein SMARCA4 expression or a function of SMARCA4 is evaluated by a method comprising:
(a) obtaining a biological sample from the subject;
(b) sequencing at least one DNA sequence encoding a SMARCA4 protein from the biological sample or a portion thereof; and
(c) determining if the at least one DNA sequence encoding a SMARCA4 protein contains a mutation affecting the expression and/or function of the SMARCA4 protein.

20.-21. (canceled)

22. The method of claim 1, wherein the subject is less than 40 years of age.

23. The method of claim 22, wherein the subject is less than 30 years of age or less than 20 years of age.

24. (canceled)

25. The method of claim 1, wherein the subject is between 20 and 30 years of age, inclusive of the endpoints.

26. The method of claim 1, wherein treating comprises preventing and/or inhibiting proliferation of a SCCOHT cell.

27. A method of treating SCCOHT in a subject in need thereof comprising administering to the subject a therapeutically effective amount of tazemetostat,
wherein the tazemetostat is formulated as an oral tablet,
wherein the therapeutically effective amount is about 800 mg/kg, and
wherein the tazemetostat is administered twice per day.
